(19) United States
(12) Patent Application Publication
(10) Pub. No.: US 2023/0062782 A1
(43) Pub. Date: Mar. 2, 2023

(54) ULTRASOUND AND STEREO IMAGING SYSTEM FOR DEEP TISSUE VISUALIZATION

(71) Applicant: Cilag GmbH International, Zug (CH)

(72) Inventors: Tarik Yardibi, Wayland, MA (US); Charles J. Scheib, Loveland, OH (US); Cortney E. Henderson, Loveland, OH (US); Steen Møller Hansen, Los Gatos, CA (US); Emir Osmanagic, Raynham, MA (US); Tamara Lanier, Cincinnati, OH (US)

(21) Appl. No.: 17/465,046

(22) Filed: Sep. 2, 2021

(51) Int. Cl. A61B 8/08; A61B 8/00; A61B 8/13; A61B 1/05; A61B 1/00; A61B 90/00

(52) U.S. Cl. CPC A61B 8/0833; A61B 8/5207; A61B 8/461; A61B 8/13; A61B 1/05; A61B 1/00193; A61B 90/39

(57) ABSTRACT

Systems and methods for deep tissue visualization using ultrasound and stereo imaging are provided. Various aspects of the present disclosure provide intraoperative identification of sub-tissue surface critical structures (e.g., identification of ureters, nerves, and/or vessels). For example, various surgical visualization systems disclosed herein can enable the visualization of one or more portions of critical structures below the surface of the tissue in an anatomical field in real-time. Such surgical visualization systems can augment the clinician's endoscopic view of an anatomical field with a virtual, real-time depiction of the critical structure as a visible image overlay on the surface of visible tissue in the field of view of the clinician.

### FIELD

The present disclosure relates generally to ultrasound and stereo imaging for deep tissue visualization.

### BACKGROUND

Surgical systems often incorporate an imaging system, which can allow the clinician(s) to view the surgical site and/or one or more portions thereof on one or more displays such as a monitor. The display(s) can be local and/or remote to a surgical theater. An imaging system can include a scope with a camera or sensor that views the surgical site and transmits the view to a display that is viewable by a clinician. Scopes include, but are not limited to, laparoscopes, arthroscopes, angioscopes, bronchoscopes, choledochoscopes, colonoscopes, cytoscopes, duodenoscopes, enteroscopes, esophagogastro-duodenoscopes (gastroscopes), endoscopes, laryngoscopes, nasopharyngo-neproscopes, sigmoidoscopes, thoracoscopes, ureteroscopes, and exoscopes.

For example, certain concealed structures, physical contours, and/or dimensions of structures within a surgical field may be unrecognizable intraoperatively by certain imaging systems. Additionally, certain imaging systems may be incapable of communicating and/or conveying certain information regarding the concealed structures to clinician(s) intraoperatively.

Accordingly, there remains a need for improved imaging techniques for deep tissue visualization.

### SUMMARY

In an aspect, a system is provided and can include an endoscope that can include an image sensor configured to acquire real-time image data characterizing an image of a tissue surface, an ultrasound probe that can include an ultrasound transducer disposed at a distal end thereof and configured to acquire real-time ultrasound data characterizing a portion of a target feature located below the tissue surface, and at least one processor in operable communication with each of the image sensor and the ultrasound transducer. The at least one processor can be configured to receive the real-time image data and the real-time ultrasound data, determine a graphical depiction, based on the received real-time ultrasound data, that characterizes the target feature, and provide a composite image that includes the real-time image data and the graphical depiction and characterizes a location of the target feature relative to the tissue surface.

In some embodiments, the at least one processor can be configured to provide the composite image to a graphical display for display thereon. In some embodiments, the real-time image data and the real-time ultrasound data can be time-correlated. In some embodiments, the real-time image data can include a visual image of the ultrasound probe, the at least one processor can be further configured to determine a location of the ultrasound probe relative to the tissue surface based on the real-time image data, and the graphical depiction can be determined based on the determined ultrasound probe location. In some embodiments, the ultrasound probe can include a marker formed on an external surface thereof, the at least one processor can be further configured to determine a position of the marker relative to the tissue surface when the marker is in a field of view of the image sensor, and the ultrasound probe location can be determined based on the determined position of the marker. In some embodiments, the endoscope can include a projector configured to project a structured light pattern onto the tissue surface and the ultrasound probe, the image sensor can be configured to acquire an image of the structured light pattern, the at least one processor can be further configured to determine a position of the ultrasound probe relative to the tissue surface based on the acquired structured light pattern image, and the composite image can be determined based on the determined position of the ultrasound probe and the acquired structured light pattern image. In some embodiments, the graphical depiction can include an ultrasound-generated image of the portion of the target feature. In some embodiments, the at least one data processor can be further configured to receive target feature data characterizing a second portion of the target feature and to determine the graphical depiction based on the target feature data. In some embodiments, the target feature data can include target feature ultrasound data characterizing the second portion of the target feature and acquired by the ultrasound transducer. In some embodiments, wherein the target feature data is acquired by a computerized tomography scanner. In some embodiments, the at least one data processor can be configured to identify the target feature based on the received real-time image data and the received real-time ultrasound data. In some embodiments, the image sensor can be a stereo camera.

In another aspect, a method is provided and can include receiving, in real time and from an image sensor of an endoscope, image data characterizing a visual image of a surgical field of interest; receiving, in real time and from an ultrasound transducer of an ultrasound probe, ultrasound data characterizing at least a portion of the surgical field of interest located below a tissue surface; determining, based on the received ultrasound data, a graphical depiction that characterizes the surgical field of interest; and providing, in real time, a composite image that includes the image data and the graphical depiction and characterizes a location of the surgical field of interest relative to the tissue surface.

In some embodiments, the portion can include a critical structure. In some embodiments, the portion can include a target feature. In some embodiments, the graphical depiction can include an ultrasound-generated image of the portion of the surgical field of interest. In some embodiments, the method can include receiving field data characterizing a second portion of the surgical field of interest, and the determining of the graphical depiction can be based on the field data. In some embodiments, the method can include identifying the surgical field of interest based on at least one of the received image data, the received ultrasound data, and the received field data. In some embodiments, the image data can characterize a visual image of the ultrasound probe, the method can include determining a location of the ultrasound probe relative to the tissue surface based on the received image data, and the location of the portion of the surgical field of interest relative to the tissue surface can be determined based on the received ultrasound data and the determined location of the ultrasound probe. In some embodiments, the method can include determining a location of the second portion of the surgical field of interest relative to the tissue surface based on the received field data and the determined location of the portion of the surgical field of interest relative to the tissue surface, and the composite image can characterize the second portion of the surgical field of interest. In some embodiments, the method can include providing the composite image to a graphical display for display thereon.

In another aspect, a system is provided and can include at least one data processor and memory storing instructions configured to cause the at least one data processor to perform operations. The operations can include receiving, in real time and from an image sensor of an endoscope, image data characterizing a visual image of a surgical field of interest; receiving, in real time and from an ultrasound transducer of an ultrasound probe, ultrasound data characterizing at least a portion of the surgical field of interest located below a tissue surface; determining, based on the received ultrasound data, a graphical depiction that characterizes the surgical field of interest; and providing, in real time, a composite image that includes the image data and the graphical depiction and characterizes a location of the surgical field of interest relative to the tissue surface.

### BRIEF DESCRIPTION OF DRAWINGS

This invention will be more fully understood from the following detailed description taken in conjunction with the accompanying drawings, in which:

FIG. 1 is a schematic of a surgical visualization system including an imaging device and a surgical device, the surgical visualization system configured to identify a critical structure below a tissue surface, according to at least one aspect of the present disclosure;

FIG. 2 is a schematic of a control system for a surgical visualization system, according to at least one aspect of the present disclosure;

FIG. 3 illustrates a composite image generated by the surgical visualization system, according to at least one aspect of the present disclosure; and

FIG. 4 illustrates one embodiment of a method of at least some implementations of the current subject matter that can provide for visualizing deep tissue using ultrasound and stereo imaging.

### DETAILED DESCRIPTION

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

Further, in the present disclosure, like-named components of the embodiments generally have similar features, and thus within a particular embodiment each feature of each like-named component is not necessarily fully elaborated upon. Additionally, to the extent that linear or circular dimensions are used in the description of the disclosed systems, devices, and methods, such dimensions are not intended to limit the types of shapes that can be used in conjunction with such systems, devices, and methods. A person skilled in the art will recognize that an equivalent to such linear and circular dimensions can easily be determined for any geometric shape. Sizes and shapes of the systems and devices, and the components thereof, can depend at least on the anatomy of the subject in which the systems and devices will be used, the size and shape of components with which the systems and devices will be used, and the methods and procedures in which the systems and devices will be used.

The figures provided herein are not necessarily to scale. Further, to the extent arrows are used to describe a direction a component can be tensioned or pulled, these arrows are illustrative and in no way limit the direction the respective component can be tensioned or pulled. A person skilled in the art will recognize other ways and directions for creating the desired tension or movement. Likewise, while in some embodiments movement of one component is described with respect to another, a person skilled in the art will recognize that other movements are possible. Additionally, although terms such as “first” and “second” are used to describe various aspects of a component, e.g., a first end and a second end, such use is not indicative that one component comes before the other. Use of terms of this nature may be used to distinguish two similar components or features, and often such first and second components can be used interchangeably. Still further, a number of terms may be used throughout the disclosure interchangeably but will be understood by a person skilled in the art.

The present disclosure is directed to a surgical visualization platform that leverages “digital surgery” to obtain additional information about a patient's anatomy and/or a surgical procedure. The surgical visualization platform is further configured to convey data and/or information to one or more clinicians in a helpful manner. For example, various aspects of the present disclosure provide improved visualization of the patient's anatomy and/or the surgical procedure. “Digital surgery” can embrace robotic systems, advanced imaging, advanced instrumentation, artificial intelligence, machine learning, data analytics for performance tracking and benchmarking, connectivity both inside and outside of the operating room (OR), and more. Although various surgical visualization platforms described herein can be used in combination with a robotic surgical system, surgical visualization platforms are not limited to use with a robotic surgical system. In certain instances, advanced surgical visualization can occur without robotics and/or with limited and/or optional robotic assistance. Similarly, digital surgery can occur without robotics and/or with limited and/or optional robotic assistance. Digital surgery is also applicable to non-robotic surgical procedures, including laparoscopic, arthroscopic, and endoscopic surgical procedures.

In some instances, a surgical system that incorporates a surgical visualization platform may enable smart dissection in order to identify and avoid critical structures. Critical structures include anatomical structures such as a vessels, including without limitation, arteries such as a superior mesenteric artery and veins such as a portal vein, lymph nodes, a urethra, a ureter, a common bile duct, and nerves such as a phrenic nerve. Other critical structures include a tumor. Critical structures can be determined on a patient-by-patient and/or a procedure-by-procedure basis. Example critical structures are further described herein. Smart dissection technology may provide improved intraoperative guidance for dissection and/or can enable smarter decisions with critical anatomy detection and avoidance technology, for example. In other instances, the surgical visualization platform can be configured to identify a foreign structure in the anatomical field, such as a surgical device, surgical fastener, clip, tack, bougie, band, and/or plate, for example.

A surgical system incorporating a surgical visualization platform may also enable smart anastomosis technologies that provide more consistent anastomoses at optimal location(s) with improved workflow. Cancer localization technologies may also be improved with the various surgical visualization platforms and procedures described herein. For example, cancer localization technologies can identify and track a cancer location, orientation, and its margins. In certain instances, the cancer localizations technologies may compensate for movement of a tool, a patient, and/or the patient's anatomy during a surgical procedure in order to provide guidance back to the point of interest for the clinician.

In some aspects, a surgical visualization platform may provide improved tissue characterization and/or lymph node diagnostics and mapping. For example, tissue characterization technologies may characterize tissue type and health without the need for physical haptics, especially when dissecting and/or placing stapling devices within the tissue. Certain tissue characterization technologies described herein may be utilized without ionizing radiation and/or contrast agents. With respect to lymph node diagnostics and mapping, a surgical visualization platform may preoperatively locate, map, and ideally diagnose the lymph system and/or lymph nodes involved in cancerous diagnosis and staging, for example.

During a surgical procedure, the information available to the clinician via the “naked eye” and/or an imaging system may provide an incomplete view of the surgical site. For example, certain structures, such as structures embedded or buried within an organ, can be at least partially concealed or hidden from view. Additionally, certain dimensions and/or relative distances can be difficult to ascertain with existing sensor systems and/or difficult for the “naked eye” to perceive. Moreover, certain structures can move preoperatively (e.g., before a surgical procedure but after a preoperative scan) and/or intraoperatively. In such instances, the clinician might be unable to accurately determine the location of a critical structure intraoperatively.

When the position of a critical structure is uncertain and/or when the proximity between the critical structure and a surgical tool is unknown, a clinician's decision-making process can be inhibited. For example, a clinician may avoid certain areas in order to avoid inadvertent dissection of a critical structure; however, the avoided area may be unnecessarily large and/or at least partially misplaced. Due to uncertainty and/or overly excessive exercises in caution, the clinician may not access certain desired regions. For example, excess caution may cause a clinician to leave a portion of a tumor and/or other undesirable tissue in an effort to avoid a critical structure even if the critical structure is not in the particular area and/or would not be negatively impacted by the clinician working in that particular area. In certain instances, surgical results can be improved with increased knowledge and/or certainty, which can allow a surgeon to be more accurate and, in certain instances, less conservative/more aggressive with respect to particular anatomical areas.

In various aspects, the present disclosure provides a surgical visualization system for intraoperative identification and avoidance of critical structures. In one aspect, the present disclosure provides a surgical visualization system that enables enhanced intraoperative decision making and improved surgical outcomes. In various aspects, the disclosed surgical visualization system provides advanced visualization capabilities beyond what a clinician sees with the “naked eye” and/or beyond what an imaging system can recognize and/or convey to the clinician. The various surgical visualization systems can augment and enhance what a clinician is able to know prior to tissue treatment (e.g., dissection) and thus may improve outcomes in various instances.

Systems and methods for deep tissue visualization using ultrasound and stereo imaging are provided. Various aspects of the present disclosure provide intraoperative identification of sub-tissue surface critical structures (e.g., identification of tumors, common bile ducts, ureters, nerves, and/or vessels (e.g., arteries, veins, etc.)). For example, various surgical visualization systems disclosed herein can enable the visualization of critical structures below the surface of the tissue in an anatomical field in real-time. Such surgical visualization systems can augment the clinician's endoscopic view of an anatomical field with a virtual, real-time depiction of the critical structure as a visible image overlay on the surface of visible tissue in the field of view of the clinician.

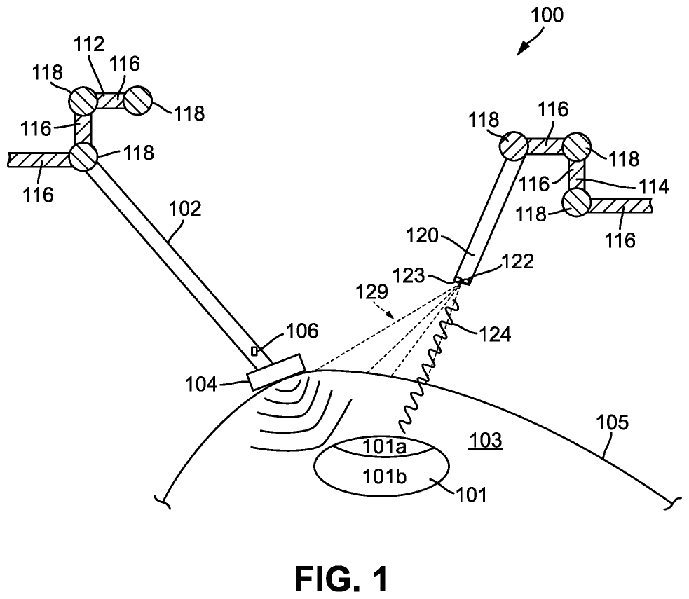

FIG. 1 is a schematic of a surgical visualization system 100 according to at least one aspect of the present disclosure. The surgical visualization system 100 can create a visual representation of a critical structure 101 within an anatomical field that is located beneath a tissue surface 105 of tissue 103 (e.g., fat, connective tissue, adhesions, and/or organs) located within the anatomical field that would be otherwise difficult or impossible to image in real-time. In certain instances, the surgical visualization system 100 can be used intraoperatively to provide real-time information to the clinician regarding locations and dimensions of critical structures during a surgical procedure. The surgical visualization system 100 is configured for intraoperative identification of critical structure(s) and/or to facilitate the avoidance of critical structure(s), such as the critical structure 101, by a surgical device. For example, by identifying the critical structure 101, a clinician can maneuver a surgical device around the critical structure 101 and/or a region in a predefined proximity of the critical structure 101 during a surgical procedure and thereby avoid inadvertent dissection of the critical structure 101. In some embodiments, the surgical visualization system 100 can create a visual representation of a foreign structure in the anatomical field, such as a surgical device, a surgical fastener, a clip, a tack, a bougie, a band, and/or a plate, for example, and, the surgical visualization system 100 can be configured for intraoperative identification of the foreign structure(s) and/or to facilitate the avoidance of the foreign structure(s) by a surgical device using the techniques described herein with respect to the intraoperative identification of the critical structure 101.

In some embodiments, the critical structure 101 can be an anatomical structure of interest. For example, the critical structure 101 can be a ureter, an artery such as a superior mesenteric artery, a vein such as a portal vein, a nerve such as a phrenic nerve, and/or a tumor, among other anatomical structures. As shown in FIG. 1, the critical structure 101 can have multiple portions, such as a first critical structure portion 101a and a second critical structure portion 101b. As shown, the first critical structure portion 101a can be located closer to the tissue surface 105 than the second critical structure portion 101b.

As mentioned above, the critical structure 101 may be embedded in tissue 103, such that the critical structure 101 may be positioned below the surface 105 of the tissue 103. In such instances, the tissue 103 can fully conceal the critical structure 101 from a clinician's view. However, in other instances, the critical structure 101 may be only partially obscured from view.

As shown in FIG. 1, the surgical visualization system 100 can include an imaging system that includes an endoscope 120 disposed in proximity to the anatomical field. The endoscope 120 can include an image sensor 122 that is disposed at a distal end of the endoscope 120 and is configured to acquire real-time image data that characterizes an image of the tissue surface 105 and/or other portions of the anatomical field of view. In some embodiments, the image sensor 122 can include a three-dimensional camera which is configured to obtain the real-time image data that characterizes a visual image of the tissue surface 105. In some embodiments, the image sensor 122 can include a spectral camera (e.g., a hyperspectral camera, multispectral camera, or selective spectral camera), which is configured to detect reflected spectral waveforms and to generate a spectral cube of images based on the molecular response of portions of the anatomical field to wavelengths of light shown on the anatomical field portions. As described in further detail below, the endoscope 120 can also include an emitter 123 configured to emit light having hyperspectral, multispectral, and/or selective spectral wavelengths to thereby illuminate the portions of the anatomical field with the emitted light, and the reflections of the emitted light can be detected by the spectral camera as described above. The use of a spectral camera and the emitter 123 configured to emit hyperspectral, multispectral, and/or selective spectral light allows for the acquisition of real-time image data characterizing a location and/or dimensions of a portion of the critical structure that is below, but proximate to, the tissue surface 105, such as the first critical structure portion 101a, as discussed in further detail below.

As mentioned above, the image sensor 122 is configured to detect light at various wavelengths, such as, for example, visible light, spectral light waves (visible or invisible), and a structured light pattern (visible or invisible). The image sensor 122 may include a plurality of lenses, sensors, and/or receivers for detecting the different signals, such that the image sensor 122 is a stereo camera. For example, the imaging device 120 can include a right-side lens and a left-side lens used together to record two two-dimensional images at the same time and thus generate a three-dimensional image of the surgical site, render a three-dimensional image of the surgical site, and/or determine one or more distances of features and/or critical structures at the surgical site. Additionally or alternatively, the image sensor 122 can be configured to receive images indicative of the topography of the visible tissue and the identification and position of hidden critical structures, as further described herein. And, in some embodiments, the field of view of the image sensor 122 can overlap with a pattern of light (e.g., structured light) projected onto the surface 105 of the tissue, such that the image sensor 122 can detect the projected structured light pattern present in the field of view.

In some embodiments, the emitter 123 of the endoscope 120 can also be configured to emit the aforementioned pattern of structured light, such as stripes, grid lines, and/or dots, to enable the determination of the topography or landscape of the surface 105 as well as the spatial position of the surface 105 in the anatomical field. For example, projected light arrays 129 can be used for three-dimensional scanning and registration on the surface 105. In one aspect, the projected light array 129 can be employed to determine the shape defined by the surface 105 of the tissue 103 and/or the motion of the surface 105 intraoperatively. The image sensor 122 is configured to detect the projected light arrays reflected from the surface 105 to determine the topography of the surface 105 and various distances with respect to the surface 105. In some embodiments, the projected light arrays 129 can be emitted from the emitter 123 such that the light arrays 129 are projected onto other surgical tools present in the anatomical field (e.g., a surgical tool having an end effector configured to manipulate/dissect tissue, an ultrasound probe (such as ultrasound probe 102, described in detail below), etc.) and within the field of view of the image sensor 122. As such, the light arrays 129 can enable the determination of the spatial position of the surgical tools in the anatomical field.

In some embodiments, as mentioned above, the emitter 123 can also include an optical waveform emitter that is configured to emit electromagnetic radiation 124 that can penetrate the surface 105 of the tissue 103 and reach the critical structure 101. The image sensor 122 disposed on the endoscope 120 is configured to detect the effect of the electromagnetic radiation received by the image sensor 122. The image sensor 122 and the optical waveform emitter of the emitter 123 may form one or more components of a multispectral imaging system and/or a selective spectral imaging system, for example. The wavelengths of the electromagnetic radiation 124 emitted by the emitter 123 can enable the identification of the type of anatomical and/or physical structure within range of the electromagnetic radiation, such as the critical structure 101, in real-time. For example, the emitter 123 can emit light at a wavelength selected such that one or more wavelengths of light is reflected off of a portion of the critical structure 101 to thereby form a spectral signature for the critical structure 101. This spectral signature can detected by the image sensor 122, and the spectral signature can be analyzed by the system 100 to thereby identify the critical structure 101 automatically and in real-time. As such, identification of the critical structure 101 can be accomplished through spectral analysis.

As shown in FIG. 1 and described herein, the image sensor 122 and the emitter 123 are positioned at the distal end of the endoscope 120, which can be positionable by the robotic arm 114, or by the surgeon in the case of a laparoscopic or open surgical procedure. However, in some embodiments, the emitter 123 can be positioned on an additional surgical tool present in the anatomical field, separate from the endoscope 120.

As referenced above and shown in FIG. 1, the surgical visualization system 100 can also include an ultrasound probe 102 that is disposed in proximity to the anatomical field. The ultrasound probe 102 can include an ultrasound transducer 104 disposed at a distal end thereof. The ultrasound transducer 104 can be configured to acquire, in real-time, ultrasound data that characterizes at least a portion of the critical structure 101. In some implementations, the ultrasound probe 102 can also include a marker 106 disposed on an exterior surface of the ultrasound probe 102. As explained in further detail below, the marker 106 can be positioned on the ultrasound probe 102, such that it is within the field of view of the image sensor 122 during a surgical procedure, to enable determination of the spatial position of the ultrasound probe 102 in the anatomical field. In some embodiments, the marker 106 can be a unique visual marker. In some embodiments, the marker 106 can be an infrared marker.

Although useful for non-robotic assisted surgical procedures such as laparoscopic and open surgeries, in one aspect, the surgical visualization system 100 may be incorporated into a robotic system. For example, the robotic system may include a first robotic arm 112 and a second robotic arm 114. The robotic arms 112, 114 include rigid structural members 116 and joints 118, which can include servomotor controls. The first robotic arm 112 is configured to maneuver the surgical device 102, and the second robotic arm 114 is configured to maneuver the imaging device 120. A robotic control unit (not shown) can be configured to issue control motions to the robotic arms 112, 114, which can affect the orientation and positioning of the ultrasound probe 102 and the imaging device 120.

In certain instances, one or more of the robotic arms 112, 114 may be separate from a main robotic system used in the surgical procedure. At least one of the robotic arms 112, 114 can be positioned and registered to a particular coordinate system without a servomotor control. For example, a closed-loop control system and/or a plurality of sensors for the robotic arms can control and/or register the position of the robotic arm(s) 112, 114 relative to the particular coordinate system. Similarly, the position of the surgical device 102 and the imaging device 120 can be registered relative to a particular coordinate system.

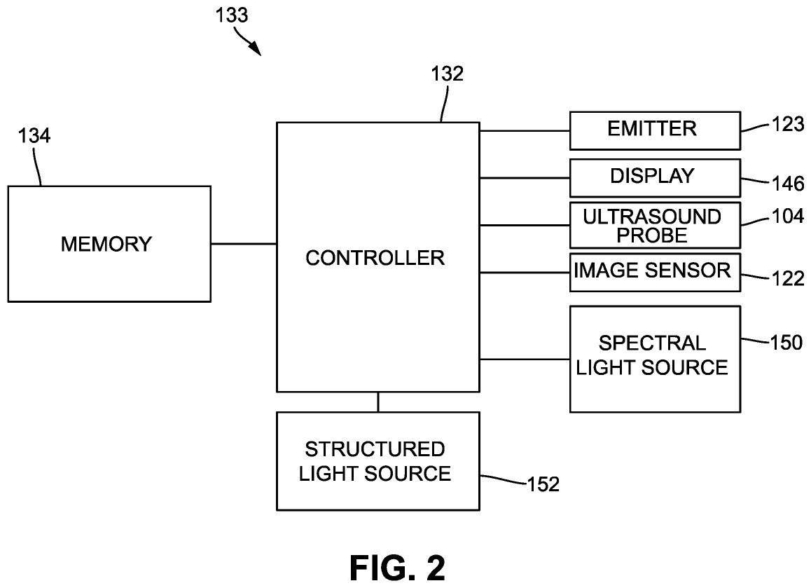

FIG. 2 is a schematic diagram of a control system 133 that can be utilized with the surgical visualization system 100. As shown, the control system 133 includes a controller 132 having at least one processor that is in operable communication with, among other components, a memory 134, the ultrasound transducer 104, the image sensor 122, the emitter 123, and a display 146. The memory 134 is configured to store instructions executable by the processor of the controller 132 to determine and/or recognize the portions of the critical structures (e.g., the critical structure 101 in FIG. 1), to determine and/or compute one or more distances of one or more portions of the critical structure 101 from the tissue surface 105, to determine and/or compute three-dimensional digital representations or graphical depictions of one or more portions of the critical structure 101, and to display the real-time image data and a graphical depiction of the real-time ultrasound data acquired by the ultrasound probe 104 and/or the image sensor 122 in a composite image on the display 146, as explained in further detail below. In some embodiments, the control system 133 can include one or more of a spectral light source 150 configured to generate wavelengths of light in the desired spectral light range for emission by emitter 123, and a structured light source 152 configured to generate wavelengths and patterns of light in the desired structured light range for emission by emitter 123. As mentioned above, in some embodiments, spectral light source 150 can be a hyperspectral light source, a multispectral light source, and/or a selective spectral light source.

As referenced above, a composite image that includes 1) an image of tissue surfaces present in the anatomical field that is characterized by the image data acquired by the image sensor 122, and 2) a graphical depiction of critical structures, such as critical structure 101, fully or partially obscured beneath the tissue surfaces, can be determined by the controller 132 by analyzing the aforementioned forms of data received from the ultrasound transducer 104 and the image sensor 122. For example, the graphical depiction can include an ultrasound-generated image of the critical structure 101 that is generated based on the ultrasound data received from the ultrasound transducer 104. The ultrasound-generated image of the critical structure 101 can be time-correlated by the controller 132 with the image data received from the image sensor 122 characterizing the visual image of the tissue surfaces (such as tissue surface 105), such that the ultrasound-generated image of the graphical depiction can be overlaid on top of the image data to form the composite image showing the image of the tissue surfaces and the critical structures together in real-time. The composite image can be provided to a graphical display for depiction thereon and viewing by a surgeon in real-time during a procedure.

Additionally or alternatively, in some embodiments, imaging data characterizing one or more portions of the critical structure 101 can be determined pre-operatively by such methods as magnetic resonance imaging (MRI) or computerized tomography (CT) scans and provided to the controller 132 for inclusion in the aforementioned composite image. For example, the pre-operative imaging data can be included in the composite image with 1) the above-described graphical depiction characterizing the ultrasound data acquired by the ultrasound transducer 104 and/or 2) the above-described image data acquired by the image sensor 122 that characterizes the surface of the surgical field and the critical structure 101 to provide an enhanced composite depiction of the surgical field and the critical structure 101 in the surgeon's view in real-time.

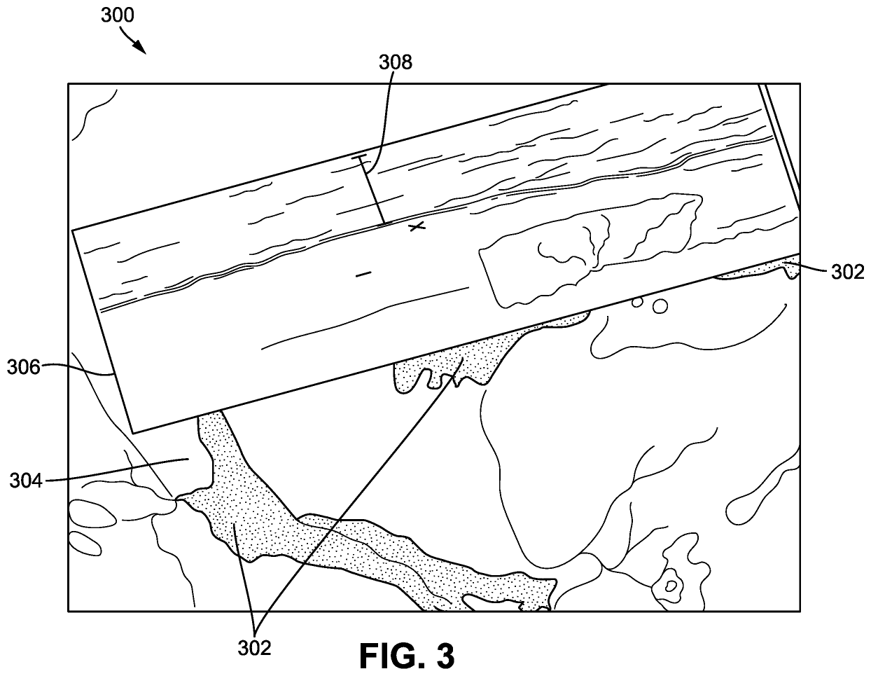

In addition, in some embodiments, graphical data characterizing the above-described spectral signatures acquired by the image sensor 122 and characterizing the critical structure 101 can be added to the composite image to provide a comprehensive graphical presentation of the data acquired by the system 100. For example, the detected spectral signature of the critical structure 101 can be used to generate a false color image characterizing a location of the critical structure 101 in the surgical field, and the false color image can be overlaid on the image of the surgical field in the composite image and presented to the surgeon to facilitate the identification of the location of the critical structure 101 within the surgical field. FIG. 3 shows an example composite image 300 of the surgical field in which a false color image 302 (the bounded portions of the composite image 300 shown in FIG. 3) is overlaid on a visual image 304 of the surgical field. As shown in FIG. 3, in some embodiments, a graphical depiction 306 characterizing the ultrasound data acquired by the ultrasound transducer 104 can be overlaid on top of the composite image 300 (including the false color image and the visual image). As shown, the graphical depiction 306 can include a depth indicator 308 that is configured to provide a graphical indication to a surgeon of the depth of the critical structure 101 relative to the tissue surface 105. Thus, the depth indicator 308 allows for the surgeon to receive depth information for the critical structure 101 in real-time.

In some embodiments, the graphical depiction (such as graphical depiction 306) can be determined by the controller 132 based on data received from the ultrasound transducer 104 that characterizes an ultrasonic image of the first critical structure portion 101a and based on data that characterizes a location of the ultrasound transducer 104 relative to the tissue surface 105 and the image sensor 122. For example, the controller 132 can determine the spatial position of the ultrasound probe 102 relative to the endoscope 120 by detecting a presence of the marker 106 on an external surface of the ultrasound probe 102 in the image data received from the image sensor 122. As the location of the marker 106 relative to the ultrasound transducer 104 is known to the controller 132, the controller 132 can determine the location of the detected marker 106 and thereby determine the location of the ultrasound transducer 104. In some embodiments, instead of using a marker such as marker 106 to locate the ultrasound probe 102, the location of the ultrasound probe 102 can be detected by the use of the structured light or spectral light techniques described elsewhere herein.

As the depth of the signal penetration of the ultrasonic signals emitted by the ultrasound transducer 104 is known to the controller 132, the controller 132 can use the determined location of the ultrasound transducer 104 with the known signal penetration depth of the ultrasound transducer 104 to determine a depth of the first critical structure portion 101a relative to the tissue surface 105. The controller 132 can then use the determined depth of the first critical structure portion 101a and the ultrasonic image data to create the graphical depiction, which can include graphical representations of the ultrasonic image of the first critical structure portion 101a and of information characterizing the spatial position of the first critical structure portion 101a relative to the tissue surface. As mentioned above, the graphical depiction can be presented with the visual image data acquired by the image sensor 122 in the composite image to aid a surgeon in identifying the location of the critical structure 101.

The graphical depiction can also be determined by the controller 132 based on data received from the ultrasound transducer 104 that characterizes an ultrasonic image of the second critical structure portion 101b. To obtain the data, the ultrasound probe 102 can be continuously moved around the critical structure 101 such that the ultrasound transducer 104 is within range of the second critical structure portion 101b, and the ultrasound transducer 104 can acquire the data characterizing the location of the second critical structure portion 101b in real time as the ultrasound probe 102 is manipulated. The controller 132 can analyze this real-time data to generate the graphical depiction, which includes a graphical representation of the second critical structure portion 101b and the information characterizing the spatial position of the second critical structure portion 101b relative to the tissue surface 105.

In some embodiments, the graphical depiction of the second critical structure portion 101b can also be determined by the controller 132 based on preoperative MRI and/or CT data characterizing the location and dimensions of the second critical structure portion 101b that is received by the controller 132. In some embodiments, the graphical depiction of the first critical structure portion 101a can also be determined by the controller 132 based on spectral image data characterizing the location and dimensions of the first critical structure portion 101a that is acquired by the image sensor 122 when spectral light is emitted from the emitter 123 in the direction of the first critical structure portion 101a.

In some embodiments, the data characterizing the first critical structure portion 101a (that is acquired and processed by the controller 132 using the techniques above to generate the graphical depiction of the first critical structure portion 101a) can be combined, by the controller 132, with the data characterizing the second critical structure portion 101b (that is acquired and processed using the techniques above to generate the graphical depiction of the second critical structure portion 101b). The combined data set can be used to generate a combined graphical depiction that includes a combined graphical representation of the first critical structure portion 101a and the second critical structure portion 101b as well as positional and dimensional information of both the first and second critical structure portions 101a, 101b. This combined graphical depiction can be combined, by the controller 132, with visual image data of the anatomical field acquired by the image sensor 122 to generate, on a real-time basis, a composite image that includes a full graphical representation of the critical structure using disparate data sets sourced from different imaging and positional data gathering modalities.

In some embodiments, the composite image 300 can be updated in real-time based on the real-time position of the ultrasound probe, as determined by the location of the marker 106 on the ultrasound probe 102 acquired by the image sensor 122, and based on the real-time ultrasound data acquired by the ultrasound transducer 104. For example, the system can use the detected location of the ultrasound probe and the real-time ultrasound data to position- and time-correlate the ultrasound data presented in graphical depiction 306 with the visual image 304 and/or the false color image 302. However, in some embodiments, if a marker is not present on the ultrasound tip, the visual image 304 can be position-and-time correlated to the graphical depiction 306 using by using registration algorithms and/or simultaneous localization and mapping (SLAM) techniques. Registration algorithms and/or SLAM techniques can also be used to position- and time-correlate the above-described pre-operative imaging data to the false color image 302, the visual image 304, and/or the graphical depiction 306. In some embodiments, the spectral signatures of the critical structures acquired by the image sensor 122 can be used to position- and time-correlate the false color image 302 to the graphical depiction 306 and thereby establish, or improve the accuracy of, the position/time correlations of the above-described components of the real-time composite image 300.

Since the position of the ultrasound probe 102 can be tracked in real time relative to the location of the endoscope 120, the relative movements between the ultrasound probe 102 and the endoscope 120 are determined by the controller 132 and used to continuously adjust and maintain the alignment of the graphical depiction relative to the visual image of the anatomical field presented beneath the graphical depiction in the composite image. This functionality provides the ability to generate a continuous stream of composite images of the anatomical field and to provide the continuous stream to a graphical display, such as display 146, for viewing, in real-time, by a surgeon in a surgical environment. In some embodiments, the composite image 300 can be presented on the display 146 in a three-dimensional (3D) viewable format, such that a surgeon wearing 3D viewing glasses can benefit from realistic depth perception when viewing the surgical field in the composite image 300. As such, a surgeon can have a real-time view of deeply embedded critical structures in a 3D camera view.

Such a surgical visualization system can determine the position of one or more critical structures without finite element analysis or predictive modeling techniques. Moreover, the three-dimensional digital representation can be generated by such a surgical visualization system in real time as the anatomical structure(s) move. The surgical visualization system can integrate preoperative images with a real-time three-dimensional model to convey additional information to the clinician intraoperatively. Additionally, the three-dimensional digital representations can provide more data than a three-dimensional camera scope, which only provides images from which a human eye can then perceive depth.

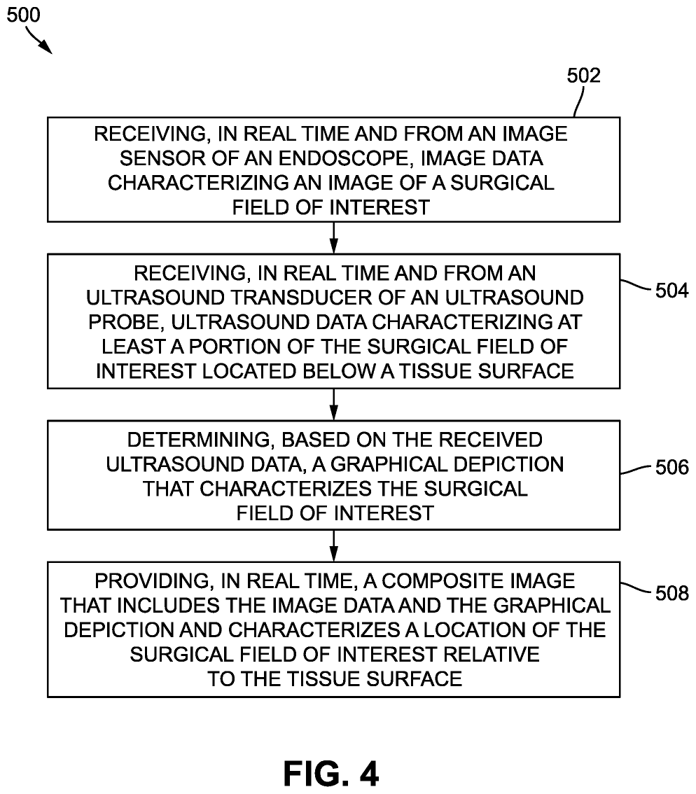

FIG. 4 illustrates one embodiment of a method 500 of at least some implementations of the current subject matter that can provide for visualizing deep tissue using ultrasound and stereo imaging. The method 500 is described with respect to the system 100 of FIGS. 1-2 and the composite image of FIG. 3, but other embodiments of systems can be similarly used.

In the method 500, at 502, image data characterizing an image of a surgical field of interest can be received from an image sensor 122 of an endoscope 120, and, at 504, ultrasound data characterizing at least a portion of the surgical field of interest located below a tissue surface can be received from an ultrasound transducer 104 of an ultrasound probe 102. In some embodiments, the portion can include the critical structure 101 located in tissue 103 below the tissue surface 105. In some embodiments, the portion can include a target feature. In some embodiments, the portion can include a section of the critical structure 101, such as the first critical structure portion 101a. In some embodiments, the received image data can characterize a visual image of the ultrasound probe 102, and a location of the ultrasound probe 102 relative to the tissue surface 105 can be determined based on the image data received from the image sensor 122. In some embodiments, the location of the portion of the surgical field of interest relative to the tissue surface can be determined based on the ultrasound data received from the ultrasound transducer 104 and the determined location of the ultrasound probe 102.

At 506, a graphical depiction, such as graphical depiction 306, that characterizes the surgical field of interest can be determined based on the received ultrasound data. In some embodiments, the graphical depiction can include an ultrasound-generated image of the portion of the surgical field of interest. In some embodiments, field data characterizing a second portion of the surgical field of interest can be received, and the graphical depiction can be determined based on the field data. In some embodiments, the second portion can include an additional section of the critical structure 101, such as the second critical structure portion 101b. In some embodiments, the field data can include field ultrasound data characterizing the second portion of the surgical field of interest and acquired by the ultrasound transducer 104. In some embodiments, the surgical field of interest can be identified based on at least one of the image data received from the image sensor 122, the ultrasound data received from the ultrasound transducer 104, and the received field data. In some embodiments, the field data can be received pre-operatively and can include MRI or CT scan data. In some embodiments, a location of the second portion of the surgical field of interest can be determined relative to the tissue surface based on the received field data and the determined location of the portion of the surgical field of interest relative to the tissue surface.

At 508, a composite image, such as composite image 300, that includes the image data (graphically shown as false color image 302 and/or visual image 304) and the graphical depiction (such as graphical depiction 306), and that characterizes a location of the surgical field of interest relative to the tissue surface, can be provided. In some embodiments, the composite image can characterize the second portion of the surgical field of interest. In some embodiments, the composite image can be provided to a graphical display, such as display 146, for display thereon.

In an example use of the surgical visualization system 100, the ultrasound probe 102 and the endoscope 120 can be used in an anatomical field to gather data in real-time characterizing a critical structure 101 during an actual surgical procedure to help a surgeon and/or other medical practitioner access and remove only unhealthy tissue without damaging healthy tissue, while also avoiding the time delay and inaccuracies of math-based critical structure location predictions. For example, the image sensor 122, which can include a stereo camera, can acquire a visual image of the tissue surface 105 and of the marker 106 located on the exterior surface of the ultrasound probe 102. The emitter 123 can also emit hyperspectral light, which can penetrate the tissue surface 105 and illuminate the first critical structure portion 101a, and the image sensor 122 can acquire a multispectral image of the light reflected off of the first critical structure portion 101a. The ultrasound transducer 104 of the ultrasound probe 102 can obtain ultrasound data characterizing an image of the second critical structure portion 101b. The acquired ultrasound data, visual image data, and multispectral image data can be combined, by controller 132, with preoperative image data of the first and/or second critical structure portions 101a, 101b that acquired by such modalities as MRI or CT scans to generate a 3D graphical depiction of the critical structure 101, and the controller 132 can overlay the graphical depiction of the critical structure 101 on the visual image of the tissue surface 105 acquired by the image sensor 122 to form a composite image of the tissue surface 105 and the critical structure 101 located below the tissue surface 105. The composite image can be displayed on the display 146, such that the surgeon can safely identify the location of critical structure 101 within the anatomical field and operate on the critical structure 101 without damaging unintended structures within the anatomical field. The composite image can be updated in real-time by the controller 132, which can 1) track the location of the marker 106 on the ultrasound probe 102 as obtained by the image sensor 122, 2) using the tracked location of the marker 106, correlate the position of the second critical structure portion 102b, as determined from the ultrasound data acquired by ultrasound transducer 104 and streamed to the controller 132, with the position of the tissue surface 105 and the first critical structure portion 102a as determined from the visual and multispectral image data acquired by the image sensor 122 and streamed to the controller 132 based on the location of the marker 106, and 3) modify the graphical depiction as presented on the visual image of the tissue surface 105 in the composite image based on the correlation. Thus, the location of the critical structure 101 can be tracked throughout the surgical procedure.

One skilled in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

What is claimed is:

1. A system, comprising:
an endoscope including an image sensor configured to acquire real-time image data characterizing an image of a tissue surface;
an ultrasound probe including an ultrasound transducer disposed at a distal end thereof and configured to acquire real-time ultrasound data characterizing a portion of a target feature located below the tissue surface; and
at least one processor in operable communication with each of the image sensor and the ultrasound transducer, the at least one processor configured to:
receive the real-time image data and the real-time ultrasound data,
determine a graphical depiction, based on the received real-time ultrasound data, that characterizes the target feature, and
provide a composite image that includes the real-time image data and the graphical depiction and characterizes a location of the target feature relative to the tissue surface.

2. The system of claim 1, wherein the at least one processor is configured to provide the composite image to a graphical display for display thereon.

3. The system of claim 1, wherein the real-time image data and the real-time ultrasound data are time-correlated.

4. The system of claim 1, wherein the real-time image data includes a visual image of the ultrasound probe, and wherein the at least one processor is further configured to determine a location of the ultrasound probe relative to the tissue surface based on the real-time image data, and wherein the graphical depiction is determined based on the determined ultrasound probe location.

5. The system of claim 4, wherein the ultrasound probe includes a marker formed on an external surface thereof, wherein the at least one processor is further configured to determine a position of the marker relative to the tissue surface when the marker is in a field of view of the image sensor, and wherein the ultrasound probe location is determined based on the determined position of the marker.

6. The system of claim 4, wherein the endoscope includes a projector configured to project a structured light pattern onto the tissue surface and the ultrasound probe, wherein the image sensor is configured to acquire an image of the structured light pattern, wherein the at least one processor is further configured to determine a position of the ultrasound probe relative to the tissue surface based on the acquired structured light pattern image, and wherein the composite image is determined based on the determined position of the ultrasound probe and the acquired structured light pattern image.

7. The system of claim 1, wherein the graphical depiction includes an ultrasound-generated image of the portion of the target feature.

8. The system of claim 1, wherein the at least one data processor is further configured to receive target feature data characterizing a second portion of the target feature and to determine the graphical depiction based on the target feature data.

9. The system of claim 8, wherein the target feature data includes target feature ultrasound data characterizing the second portion of the target feature and acquired by the ultrasound transducer.

10. The system of claim 8, wherein the target feature data is acquired by a computerized tomography scanner.

11. The system of claim 1, wherein the at least one data processor is configured to identify the target feature based on the received real-time image data and the received real-time ultrasound data.

12. The system of claim 1, wherein the image sensor is a stereo camera.

13. A method, comprising:
receiving, in real time and from an image sensor of an endoscope, image data characterizing an image of a surgical field of interest;
receiving, in real time and from an ultrasound transducer of an ultrasound probe, ultrasound data characterizing at least a portion of the surgical field of interest located below a tissue surface;
determining, based on the received ultrasound data, a graphical depiction that characterizes the surgical field of interest; and
providing, in real time, a composite image that includes the image data and the graphical depiction and characterizes a location of the surgical field of interest relative to the tissue surface.

14. The method of claim 13, wherein the portion includes a critical structure.

15. The method of claim 13, wherein the portion includes a target feature.

16. The method of claim 13, wherein the graphical depiction includes an ultrasound-generated image of the portion of the surgical field of interest.

17. The method of claim 13, further comprising:
receiving field data characterizing a second portion of the surgical field of interest, and wherein the determining of the graphical depiction is based on the field data.

18. The method of claim 17, further comprising:
identifying the surgical field of interest based on at least one of the received image data, the received ultrasound data, and the received field data.

19. The method of claim 17, wherein the field data includes field ultrasound data characterizing the second portion of the surgical field of interest and acquired by the ultrasound transducer.

20. The method of claim 17, wherein the image data characterizes a visual image of the ultrasound probe, and further comprising:
determining a location of the ultrasound probe relative to the tissue surface based on the received image data, and
wherein the location of the portion of the surgical field of interest relative to the tissue surface is determined based on the received ultrasound data and the determined location of the ultrasound probe.

21. The method of claim 20, further comprising:
determining a location of the second portion of the surgical field of interest relative to the tissue surface based on the received field data and the determined location of the portion of the surgical field of interest relative to the tissue surface, and
wherein the composite image characterizes the second portion of the surgical field of interest.

22. The method of claim 13, further comprising:
providing the composite image to a graphical display for display thereon.

23. A system, comprising:
at least one data processor; and
memory storing instructions configured to cause the at least one data processor to perform operations comprising:
receiving, in real time and from an image sensor of an endoscope, image data characterizing an image of a surgical field of interest;
receiving, in real time and from an ultrasound transducer of an ultrasound probe, ultrasound data characterizing at least a portion of the surgical field of interest located below a tissue surface;
determining, based on the received ultrasound data, a graphical depiction that characterizes the surgical field of interest; and
providing, in real time, a composite image that includes the image data and the graphical depiction and characterizes a location of the surgical field of interest relative to the tissue surface.
